(19) United States
(12) Patent Application Publication
(10) Pub. No.: US 2012/0201824 A1
(43) Pub. Date: Aug. 9, 2012

(54) Compositions and Methods for Inhibiting an Oncogenic Protein to Enhance Immunogenicity

(76) Inventor: Mariusz Wasik, Ardmore, PA (US)

(21) Appl. No.: 13/128,828

(22) PCT Filed: Dec. 8, 2011
(86) PCT No.: PCT/US11/64011

§ 371 Date: Apr. 25, 2012

Related U.S. Application Data
(60) Provisional application No. 61/113,455, filed on Nov. 11, 2008.

(51) Int. Cl. A61K 39/395; C12N 15/85; C07K 16/18; C07K 2/00; A61K 31/713; A61K 38/02; A61P 35/00; C12N 5/09; C12Q 1/02; A61K 39/00; A61P 37/04; C12Q 1/68; G01N 33/566; C40B 30/04; C07H 21/02

(52) U.S. Cl. 424/138.1; 536/24.5; 435/320.1; 530/387.7; 530/300; 514/44 A; 514/44 R; 514/19.3; 435/375; 435/29; 424/277.1; 435/6.12; 435/7.23; 506/9

(57) ABSTRACT

The present invention includes compositions and methods for inhibiting an oncogenic protein or its down-stream effector protein to suppress expression of a cell-surface protein involved in inhibiting immune response against malignant cells thereby enhancing immunogenicity of a cell. The invention includes inhibitors of expression of CD274 and/or its functional cell-membrane bound immunosuppressive analog. The invention includes inhibitors of function or expression of oncogenic ALK tyrosine kinase and/or other oncogenic proteins responsible for induction of expression of CD274 or its functional immunosuppressive equivalent. The invention includes inhibitors of function or expression of STAT3 and/or other cell signal transmitters and/or transcription factors activated by ALK or its functional analog involved in induction of expression of CD274 or its functional analog.

### BACKGROUND OF THE INVENTION

Anaplastic large cell lymphoma tyrosine kinase or Anaplastic Lymphoma Kinase (ALK) is a receptor tyrosine kinase (RTK) belonging to the insulin receptor subfamily. Expression of ALK has been found in a variety of malignant tumors including T/null- and B-cell lymphomas, soft tissue tumors, lung carcinomas, and neuroblastomas. ALK is present in tumors as a result of various expression mechanisms, foremost chromosomal translocations. Activation of ALK occurs either by binding of the natural ALK ligands (e.g. pleiotrophin), ALK activating point mutations or by self-aggregation of the ALK fusion proteins, which causes autophosphorylation resulting in an increase of receptor dependent signaling. ALK activation causes increased cell proliferation and apoptosis via activation of the cell signaling pathways including PKC, MAPK, STAT3, STAT5B, and PI3K/AKT.

T/null-cell lymphomas (TCL) that express ALK (ALK+ TCL) comprise a distinct category of lymphomas (Li et al., 2008, Med Res Rev 28: 372-412; Chiarle et al., 2008, Nat Rev Cancer 8: 11-23). Ectopic expression of ALK results in the affected CD4+ T lymphocytes from chromosomal translocations involving the ALK gene and several different partners, most frequently the nucleophosmin (NPM) gene (Morris et al., 1994, Science 263: 1281-1284; Shiota et al., 1994, Oncogene 9: 1567-1574). The NPM/ALK chimeric protein is not only constitutively expressed but is also chronically activated through autophosphorylation (Shiota et al., 1994, Oncogene 9: 1567-1574; Morris et al., 1997, Oncogene 14: 2175-2188). NPM/ALK displays potent cell-transforming properties as demonstrated both in vitro (Fujimoto et al., 1996, Proc Natl Acad Sci USA 93: 4181-4186; Bischof et al., 1997, Mol Cell Biol 17: 2312-2325) and in vivo (Kuefer et al., 1997, Blood 90: 2901-2910; Chiarle et al., 2003, Blood 101: 1919-1927). NPM/ALK mediates its oncogenicity by activating a number of cell-signaling proteins, including STAT3 (Li et al., 2008, Med Res Rev 28: 372-412; Chiarle et al., 2008, Nat Rev Cancer 8: 11-23; Zhang et al., 2002, J Immunol 168: 466-474). The continuous activation of these signal transmitters leads to persistent expression of genes, the protein products of which are involved in key cell functions such as the promotion of cell proliferation and protection from apoptosis.

CD279, or programmed cell death 1 (PD-1), is an immunosuppressive cell-surface receptor expressed by a subset of normal activated CD4+ and CD8+ T lymphocytes (Dong et al., 2003, J Mol Med 81: 281-287; Okazaki et al., 2007, Int Immunol 19: 813-824; Keir et al., 2008, Annu Rev Immunol 26:677-704). CD279 transduces the inhibitory signal when engaged simultaneously with the antigen T-cell receptor (TCR)-CD3 complex. CD279 has two known ligands: CD274 (also called PD-L1 or B7-H1) and CD273 (PD-L2 or B7-DC). Interactions between CD279 and its ligands control the induction and maintenance of peripheral T-cell tolerance during normal immune responses. They are also involved in immune evasion in malignancy, since cells of various tumor types have been shown to aberrantly express CD274 and, seemingly to a lesser degree, CD273.

There have been many attempts made to use various agents in immunotherapy to stimulate the immune response in a mammal, for example stimulating anti-tumor immunity in a cancer patient. There is a need in the art for the development of successful therapeutic vaccines and immunotherapies for cancer. The present invention satisfies the need in the art for development of new approaches for efficient means to induce a vigorous anti-tumor immune response.

### BRIEF SUMMARY OF THE INVENTION

The invention provides a composition for enhancing the immunogenicity of a cell. Preferably, the cell is a cancer cell

In one embodiment, the composition comprises an inhibitor of an oncogenic protein or a down-stream effector protein thereof, wherein the oncogenic protein or down-stream effector protein thereof induces directly or through the effector protein expression of an immunosuppressor.

In one embodiment, the immunosuppressor is a cell-surface suppressor of immune system response to malignant cells. Preferably, the immunosuppressor is CD274 or a functional equivalent thereof.

In another embodiment, the oncogenic protein is Anaplastic Lymphoma Kinase (ALK) or an oncogenic functional equivalent thereof capable of inducing expression of an immunosuppressor.

In yet another embodiment, the induction of expression of an immunosuppressor is through an ALK down-stream effector, a cell signal transmitter, and the gene trascription activator STAT3 or a functional equivalent thereof.

In one embodiment, the inhibitor is selected from the group consisting of a small interfering RNA (siRNA), a microRNA, an antisense nucleic acid, a ribozyme, an expression vector encoding a transdominant negative mutant, an antibody, a peptide and a small molecule.

The invention also provides an isolated cell having inhibited immunogenicity, wherein the cell contains an oncogenic protein or a down-stream effector protein thereof, further wherein the oncogenic protein or a down-stream effector protein thereof induces expression of an immunosuppressor.

In one embodiment, the oncogenic protein is ALK or a functional equivalent thereof.

In another embodiment, the induction of expression of an immunosuppressor is through STAT3 or a functional equivalent thereof.

In yet another embodiment, the immunosuppressor is a cell-surface suppressor of immune system response to malignant cells. Preferably, the immunosuppressor is CD274 or a functional equivalent thereof.

The invention provides a method of stimulating an immune response in a mammal. The method comprises administering to the mammal an effective amount of a composition comprising an inhibitor of an oncogenic protein or a down-stream effector protein thereof, wherein the oncogenic protein or down-stream effector protein thereof induces directly or through the down-stream effector expression of an immunosuppressor.

In one embodiment, the mammal is suffering from cancer. Preferably, the mammal is a human.

The invention provides a method of treating diseases or disorders associated with uncontrolled, abnormal, and/or unwanted cellular activities. The method comprises administering to a mammal in need thereof, a therapeutically effective amount of the compound or the pharmaceutical composition comprising an inhibitor of an oncogenic protein or a down-stream effector thereof, wherein the oncogenic protein or down-stream effector thereof induces directly or through the effector expression of an immunosuppressor.

In one embodiment, the method comprises administering the composition of the invention in combination with a therapeutic agent. In one aspect, the therapeutic agent is selected from the group consisting of an anti-tumor agent, a chemotherapeutic agent, an anti-cell proliferation agent, an anti-tumor vaccine and any combination thereof.

In another embodiment, the therapeutic agent is administered simultaneously, prior to, or after administration of the compound of the invention.

The invention provides a method of screening for an inhibitor of an oncogenic protein or a down-stream effector thereof, wherein the oncogenic protein or down-stream effector thereof induces directly or through the down-stream effector expression of an immunosuppressor. The method comprises contacting the inhibitor with a cell and determining the effect of the inhibitor on expression level of CD274 or a functional equivalent thereof.

In one embodiment, the method comprises determining the effect of the inhibitor on the cell concentration of CD274 or a functional equivalent thereof.

In another embodiment, the method comprises determining the immunogenicity of the cell.

The invention provides a method of diagnosing a disease in a mammal, the method comprising measuring the expression level of CD274 or a functional equivalent thereof from a biological sample derived from the mammal and comparing the expression level of CD274 or a functional equivalent thereof from a biological sample derived from an otherwise identical healthy mammal, wherein an increase in expression level of CD274 is an indication that said mammal has a disease.

In one embodiment, the biological sample is selected from the group consisting of a tumor tissue or a bodily fluid. In yet another embodiment, the bodily fluid is peripheral blood or urine.

The invention provides a method of monitoring a response to anti-cancer therapy in a mammal. The method comprises measuring the expression level of CD274 or a functional equivalent thereof from a biological sample derived from the mammal and comparing the expression level of CD274 from a biological sample derived from an otherwise identical healthy mammal, wherein a decrease in expression level of CD274 or a functional equivalent thereof is an indication that the mammal has responded to the therapy.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1E is a series of images depicting CD274 expression by ALK+ TCL cells.

FIG. 1A indicates that expression of CD274 (PD-L1) and the functionally-related CD273 (PD-L2) and CD279 (PD-1) in ALK+ TCL cell lines (SUDHL-1, and SUP-M2) exposed for 6 hr to 175 nM of an ALK inhibitor CEP-14083. IL-2-dependent, CTCL-derived Sez-4 cell line depleted of IL-2 for 16 hrs and subsequently exposed for 4 h to IL-2 or medium, served as an additional control. The results are depicted as a fold change in the hybridization signal upon cell treatment with the ALK inhibitor or IL-2 as compared to untreated cells.

FIG. 1B is an image depicting expression of CD274 mRNA in five ALK+ TCL and two CTCL cell lines as measured by RT-PCR. Expression of actin served as a positive control.

FIGS. 1C and 1D are images depicting expression of CD274 protein and CD279 protein, respectively, at the cell surface of the ALK+ TCL and CTCL cell lines detected by flow cytometry. Staining with an isotype-matched antibody of unrelated specificity served as a negative control. Jurkat cell line served as a positive control of CD279 expression.

FIG. 1E is an image depicting the expression of the CD274 protein in ALK+ TCL (SUDHL-1, SUP-M2, JB6, Karpass 299 and L-82) and two CTCL cell lines (Sez-4 and MyLa3675) examined by flow cytometry. Staining with a CD274 non-immune, isotype-matched antibody (ISO) served as a negative control.

FIG. 2, comprising FIGS. 2A through 2C, is a series of images depicting expression of CD274 in ALK+ TCL tissues. Section of lymph nodes were examined microscopically using an intermediate (100×; the large images) and high (500 and 400×; the insets) power magnification. FIG. 2A is an image of H&E staining showing predominance of large, frequently highly atypical cells. Immunohistochemical examination revealed strong, selective staining of the atypical cells by both anti-ALK (FIG. 2B) and anti-CD274 antibodies (FIG. 2C). The depicted images are representative for the eighteen ALK+ TCL cases examined.

FIGS. 2D and 2E are images demonstrating the cack of effect of mTORC1, PI3K, ERK1/2, and Jak3 inhibition on CD274 expression by the ALK+ TCL cells. ALK+ TCL SUDHL-1 cells were treated with rapamycin, wortmaninn, U0126, Jak3 inhibitor or, as a control, CEP-14083 ALK inhibitor at the depicted pre-tested highly effective concentrations and evaluated for CD274 protein expression by flow cytometry (FIG. 2D) and CD274 mRNA by RT-PCR (FIG. 2E).

FIG. 3, comprising FIGS. 3A through 3C, is a series of images demonstrating that the expression of CD274 is induced by NPM/ALK.

FIG. 3A depicts expression of CD274 mRNA in the ALK+ TCL SUDHL-1 cell line before and after treatment with 175 nM of the ALK inhibitor CEP-14083 or its structural analog CEP-11988 non-inhibitory for ALK (ALK non-inh).

FIG. 3B is an image depicting expression of CD274 protein in the ALK+ TCL cell lines before and after treatment with the ALK inhibitor CEP-14083. Treatment of the SUDHL-1 cell line with the ALK noninhibitory analog CEP-11988 served as a control.

FIG. 3C is an image depicting expression of CD274 in the IL-3-dependent BaF3 cells transfected with the intact, enzymatically active NPM/ALK, kinaseactivity negative K210R NPM/ALK mutant (ALK-KN), or empty vector after IL-3 depletion for 72 h followed by exposure for 24 h to IL-3 or medium alone (−) examined by flow cytometry. Cells labeled with an isotype-matched (ISO) rather than the anti-CD274 antibody, served as negative controls.

FIG. 4, comprising FIGS. 4A through 4 is a series of images demonstrating that NPM/ALK induces CD274 expression through STAT3.

FIG. 4A is an image depicting the effect of the siRNA-mediated STAT3 and STAT5B depletion on the CD274 mRNA expression. ALK+ TCL cell line SUDHL-1 was treated with siRNA specific for STAT3 or STAT5, STAT3/STAT5 siRNA combination, or control non-specific siRNA and evaluated by RT-PCR for expression of mRNA coding for CD274 and the depicted other molecules serving as controls.

FIG. 4B is an image depicting the effect of the siRNA-mediated STAT3 depletion on expression of the CD274 protein. SUDHL-1 cells treated with the STAT3, STAT5, STAT3/STAT5, or control siRNA were examined for CD274 protein expression by flow cytometry.

FIG. 4C is an image depicting binding of STAT3 to the CD274 gene promoter in vitro. The nuclear protein extracts from SUDHL-1 cells were incubated with the “hot”, biotin-labeled oligonucleotide probes corresponding to either of the two STAT3 binding sites identified within the CD274 gene promoter and analyzed in gel electromobility shift assay (EMSA). Extract of the SUDHL-1 cells pre-incubated with the corresponding unlabeled “cold” probes served as control.

FIG. 4D is an image depicting the binding of STAT3 to the CD274 gene promoter in vivo. Protein cell lysates from the SUDHL-1 cell line were analyzed in the ChIP assay using an anti-STAT3 rabbit polyclonal antibody and primer pairs specific for CD274 gene promoter. Non-immunoprecipitated lysates (input) and immunoprecipitates obtained with the STAT3 non-immune entire IgG rabbit serum fraction served as a positive and negative control, respectively.

### DETAILED DESCRIPTION OF THE INVENTION

The invention provides for compositions and methods for regulating Anaplastic Lymphoma Kinase (ALK). The invention is based on the discovery that malignant cell transformation caused by the oncogenic ALK is directly linked to induced expression of the immunosuppressive cell-surface protein CD274 (PD-L1, B7-H1). The CD274 expression is dependent on the expression and enzymatic activity of ALK through activation of its key signal transmitter, transcription factor STAT3.

Accordingly, the invention provides compositions and methods for targeting ALK and STAT3 and their functional equivalents in cells that express CD274 and/or similar immunosuppressive cell-surface proteins for regulating the immunogenicity of the cell. That is, the invention is based on the discovery of the direct link between an oncoprotein and expression of an immunosuppressive cell surface protein. By inhibiting the given oncoprotein and/or its key transmitter, immunogenicity of a malignant cell can be enhanced by inhibiting its expression of immunosuppressive protein.

The present invention relates to enhancing the immunogenicity of a cell by modulating an oncogenic protein and/or downstream targets and, consequently, inhibiting expression of an immunsuppressor protein in a cell. In one embodiment, the invention includes enhancing the immunogenicity of a cell by inhibiting ALK and/or STAT3, in order to inhibit expression of CD274 in a cell. The present invention indicates that vaccines and other therapies in which the immunogenicity of a cell is enhanced by modulating of the axis of NPM/ALK, STAT3 or CD274 and their functional equivalents. In addition, the present invention also provides a mechanism for breaking self tolerance in tumor vaccination. Therefore the present invention indicates a therapeutic benefit of enhancing the immunostimulatory capacity of the cell by interfering with immunosuppression in a cell.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Allogeneic” refers to a graft derived from a different animal of the same species.

“Alloantigen” is an antigen that differs from an antigen expressed by the recipient.

The term “ALK” includes the human ALK protein encoded by the ALK (Anaplastic Lymphoma Kinase) gene which in its native form is a membrane-spanning protein tyrosine kinase (PTK)/receptor.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and'humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded soley by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “DNA” as used herein is defined as deoxyribonucleic acid.

As used herein, an “effector cell” refers to a cell which mediates an immune response against an antigen. An example of an effector cell includes, but is not limited to a T cell and a B cell.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “helper Tcell” as used herein is defined as an effector Tcell whose primary function is to promote the activation and functions of other B and T lymphocytes and or macrophages. Most helper T cells are CD4 T-cells.

The term “heterologous” as used herein is defined as DNA or RNA sequences or proteins that are derived from the different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

As used herein, “immunogen” refers to a substance that is able to stimulate or induce a humoral antibody and/or cell-mediated immune response in a mammal.

The term “immunoglobulin” or “Ig”, as used herein is defined as a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “modulate” is meant to refer to any change in biological state, i.e. increasing, decreasing, and the like. For example, the term “modulate” refers to the ability to regulate positively or negatively the expression or activity of CD274, including but not limited to transcription of CD274 mRNA, stability of CD274 mRNA, translation of CD274 mRNA, stability of CD274 polypeptide, CD274 post-translational modifications, or any combination thereof. Further, the term modulate can be used to refer to an increase, decrease, masking, altering, overriding or restoring of activity, including but not limited to, CD274 activity associated with immunogenicity of a cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “polypeptide” as used herein is defined as a chain of amino acid residues, usually having a defined sequence. As used herein the term polypeptide is mutually inclusive of the terms “peptide” and “protein”.

“Proliferation” is used herein to refer to the reproduction or multiplication of similar forms of entities, for example proliferation of a cell. That is, proliferation encompasses production of a greater number of cells, and can be measured by, among other things, simply counting the numbers of cells, measuring incorporation of ³H-thymidine into the cell, and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

The term “T-cell” as used herein is defined as a thymus-derived cell that participates in a variety of cell-mediated immune reactions.

The term “B-cell” as used herein is defined as a cell derived from the bone marrow and/or spleen. B cells can develop into plasma cells which produce antibodies.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

The terms “treat,” “treating,” and “treatment,” refer to therapeutic or preventative measures described herein. The methods of “treatment” employ administration to a subject, in need of such treatment, a composition of the present invention, for example, a subject having a disorder mediated by ALK or other oncoprotein or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “ALK-mediated disorder” refers to disease states and/or symptoms associated with ALK-mediated cancers or tumors. In general, the term “ALK-mediated disorder” refers to any disorder, the onset, progression or the persistence of the symptoms of which requires the participation of ALK. Exemplary ALK-mediated disorders include, but are not limited to, cancer.

As used herein, an “oncogenic protein” refers to a protein that causes cancer. In some instances, activation of an oncogenic protein increase the chance that a normal cell will develop into a tumor cell. Non-limiting examples of an oncogenic protein is the NPM/ALK tyrosine kinase or other forms of oncogenic ALK, other chimeric tyrosine kinases, other oncogenic kinase, any other proteins responsible for induction of expression of CD274 or its functional cell-membrane immunosuppressive analog in malignant cells.

The term “effector of oncogenic protein” refers to the down-stream effectors following activation of an oncogenic protein. Non-limiting examples of an effector of oncogenic protein is a cell signal transmitter, the gene transcription activator STAT3, other STAT protein, a transcription activator activated by an oncogenic protein that is involved in induction of expression of CD274 or its functional immunosuppressive analog.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

The term “vaccine” as used herein is defined as a material used to provoke an immune response after administration of the material to a mammal.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Xenogeneic” refers to a graft derived from an animal of a different species.

### DESCRIPTION

The present invention provides compounds and methods for modulating Anaplastic Lymphoma Kinase (ALK) activity and methods of treating diseases mediated by activity of ALK and functionally similar oncoprotein using the compounds of the invention. The invention also provides compounds and methods of modulating downstream targets of ALK and its functional equivalents. Diseases mediated by ALK and functionally similar oncoproteins include, but are not limited to, diseases characterized in part by abnormalities in cell proliferation (i.e. tumor growth), programmed cell death (apoptosis), cell migration and invasion, and angiogenesis associated with tumor growth.

The present invention is based on the discovery that an oncogenic protein is able to induce expression of a cell surface immunosuppressive protein. Preferably, the oncogenic protein is an oncogenic kinase, a fused tyrosine kinase, or other forms of oncogenic ALK. More preferably, the oncogenic protein is a form of ALK that induces expression of an immunosuppressant such as CD274 or a functional immunosuppressive equivalent. In some instances, the expression of the immunosuppressant is through the STAT3 transcription factor. However, the invention should not be limited to STAT3. Rather, any transcription factor that regulates the expression of CD274 and its functional equivalents is included in the invention.

The results presented herein demonstrate that that CD274 is universally expressed in NPM/ALK expressing T-cell lymphomas. CD274 expression is induced by NPM/ALK through STAT3. The activated STAT3 acts as transcriptional activator of the CD274 gene. The disclosure presented herein demonstrates a new role for NPM/ALK and STAT3 in inducing tumor immune evasion and controlling expression of an immunosuppressive cell surface protein. Accordingly, the invention includes compositions and methods for targeting NPM/ALK, STAT3 and/or CD274 for drug therapy. In some instances, inhibiting NPM/ALK, STAT3 and/or CD274 is useful in increasing the immunogenicity of a cell and therefore allowing the immune system to respond to the cell. In addition, the invention includes monitoring CD274 expression as a diagnostic, prognostic, and/or therapy response marker.

### Compositions

As described elsewhere herein, the invention is based on the discovery that inhibition of ALK activity or expression of its key cell signal transmitter STAT3 inhibits expression of CD273. This observation is the first of its kind by providing a direct link between function of an oncogenic protein and expression of a cell-surface bound immunosuppressive protein. As such it indicates that therapeutic inhibition in cancer patients of an oncogenic protein such as ALK and/or its down-stream effector protein such as signal transmitter and transcription activator STAT3 may be beneficial, in addition to other effects, by inhibiting expression of cell-surface immunosuppressive protein such as CD274. The results presented herein also provide for combining any immunotherapy protocols in cancer with inhibitors targeting an oncogenic protein and/or its key signal transmitter(s).

The present invention relates to the discovery that inhibition of any one or more of NPM/ALK, STAT3 or CD274 provides a therapeutic benefit. Thus, the invention comprises compositions and methods for modulating any of these proteins in cell thereby enhancing immunogenicity of the cell.

Based on the disclosure herein, the present invention includes a generic concept for inhibiting an oncogenic protein or any component of the signal transduction pathway associated with the induced expression of CD274 or a functional equivalent thereof. Preferably, the signal transduction pathway includes NPM/ALK, STAT3 and/or CD274, inhibiting any one or more of these proteins is associated with increasing the immunogenicity of the cell.

In one embodiment, the invention comprises a composition for enhancing the immunogenicity of a cell. The composition comprises an inhibitor of any one or more of the following regulators: NPM/ALK, STAT3 or CD274. The composition comprising the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the siRNA polynucleotides are also intended to describe the corresponding RNA sequences and their complements, given the well established principles of complementary nucleotide base-pairing.

Preferred siRNA polynucleotides comprise double-stranded polynucleotides of about 18-30 nucleotide base pairs, preferably about 18, about 19, about 20, about 21, about 22, about 23, about 24, about 25, about 26, or about 27 base pairs, and in other preferred embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides, may include one or more siRNA polynucleotide molecules that may differ (e.g., by nucleotide insertion or deletion) in length by one, two, three, four or more base pairs as a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In preferred embodiments, the siRNA polynucleotides are homogeneous with respect to a specific nucleotide sequence.

Based on the present disclosure, it should be appreciated that the siRNAs of the present invention may effect silencing of the target polypeptide expression to different degrees. The siRNAs thus must first be tested for their effectiveness. Selection of siRNAs are made therefrom based on the ability of a given siRNA to interfere with or modulate the expression of the target polypeptide. Accordingly, identification of specific siRNA polynucleotide sequences that are capable of interfering with expression of a desired target polypeptide requires production and testing of each siRNA. The methods for testing each siRNA and selection of suitable siRNAs for use in the present invention are fully set forth herein the Examples. Since not all siRNAs that interfere with protein expression will have a physiologically important effect, the present disclosure also sets forth various physiologically relevant assays for determining whether the levels of interference with target protein expression using the siRNAs of the invention have clinically relevant significance.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

One skilled in the art will appreciate, based on the disclosure provided herein, that one way to decrease the mRNA and/or protein levels of NPM/ALK, STAT3 and/or CD274 in a cell is by reducing or inhibiting expression of the nucleic acid encoding the regulator. Thus, the protein level of the regulator in a cell can also be decreased using a molecule or compound that inhibits or reduces gene expression such as, for example, an antisense molecule or a ribozyme.

In a preferred embodiment, the modulating sequence is an antisense nucleic acid sequence which is expressed by a plasmid vector. The antisense expressing vector is used to transfect a mammalian cell or the mammal itself, thereby causing reduced endogenous expression of a desired regulator in the cell. However, the invention should not be construed to be limited to inhibiting expression of a regulator by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a regulator may be designed by incorporating target sequences into the basic ribozyme structure which are complementary to the mRNA sequence of the desired regulator of the present invention, including but are not limited to, NPM/ALK, STAT3, CD274 and equivalents thereof. Ribozymes targeting the desired regulator may be synthesized using commercially available reagents (Applied Biosystems, Inc., Foster City, Calif.) or they may be genetically expressed from DNA encoding them.

In another aspect of the invention, the regulator can be inhibited by way of inactivating and/or sequestering the regulator. As such, inhibiting the effects of a regulator can be accomplished by using a transdominant negative mutant. Alternatively an antibody specific for the desired regulator, otherwise known as an antagonist to the regulator may be used. In one embodiment, the antagonist is a protein and/or compound having the desirable property of interacting with a binding partner of the regulator and thereby competing with the corresponding wild-type regulator. In another embodiment, the antagonist is a protein and/or compound having the desirable property of interacting with the regulator and thereby sequestering the regulator.

### Antibodies

As will be understood by one skilled in the art, any antibody that can recognize and bind to an antigen of interest is useful in the present invention. That is, the antibody can inhibit in cancer patients an oncogenic protein such as ALK and/or its down-stream effector protein such as a signal transmitter and transcription activator STAT3 to provide a beneficial effect, in addition to other effects, by inhibiting expression of cell-surface immunosuppressive protein such as CD274.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Modification of Nucleic Acid Molecules

Inhibition of NPM/ALK or STAT3 or their functional equivalents, resulting in inhibition of expression of CD274, or its functional equivalent can be accomplished using a nucleic acid molecule. For example, the inhibitor is selected from the group consisting of a small interfering RNA (siRNA), a microRNA, an antisense nucleic acid, a ribozyme, an expression vector encoding a transdominant negative mutant, and the likes.

By way of example, modification of nucleic acid molecules is described in the context of an siRNA molecule. However, the methods of modifying nucleic acid molecules can be applied to other types of nucleic acid based inhibitors of the invention.

Polynucleotides of the siRNA may be prepared using any of a variety of techniques, which are useful for the preparation of specifically desired siRNA polynucleotides. For example, a polynucleotide may be amplified from a cDNA prepared from a suitable cell or tissue type. Such a polynucleotide may be amplified via polymerase chain reaction (PCR). Using this approach, sequence-specific primers are designed based on the sequences provided herein, and may be purchased or synthesized directly. An amplified portion of the primer may be used to isolate a full-length gene, or a desired portion thereof, from a suitable DNA library using well known techniques. A library (cDNA or genomic) is screened using one or more polynucleotide probes or primers suitable for amplification. Preferably, the library is size-selected to include larger polynucleotide sequences. Random primed libraries may also be preferred in order to identify 5′ and other upstream regions of the genes. Genomic libraries are preferred for obtaining introns and extending 5′ sequences. The siRNA polynucleotide contemplated by the present invention may also be selected from a library of siRNA polynucleotide sequences.

For hybridization techniques, a partial polynucleotide sequence may be labeled (e.g., by nick-translation or end-labeling with ³²P) using well known techniques. A bacterial or bacteriophage library may then be screened by hybridization to filters containing denatured bacterial colonies (or lawns containing phage plaques) with the labeled probe (see, e.g., Sambrook et al., Molecular Cloning: A Laboratory Manual, Cold Spring Harbor Laboratories, Cold Spring Harbor, N.Y., 2001). Hybridizing colonies or plaques are selected and expanded, and the DNA is isolated for further analysis.

Alternatively, numerous amplification techniques are known in the art for obtaining a full-length coding sequence from a partial cDNA sequence. Within such techniques, amplification is generally performed via PCR. One such technique is known as “rapid amplification of cDNA ends” or RACE (see, e.g., Sambrook et al., Molecular Cloning: A Laboratory Manual, Cold Spring Harbor Laboratories, Cold Spring Harbor, N.Y., 2001).

A number of specific siRNA polynucleotide sequences useful for interfering with target polypeptide expression are presented in the Examples, the Drawings, and in the Sequence Listing included herein. siRNA polynucleotides may generally be prepared by any method known in the art, including, for example, solid phase chemical synthesis. Modifications in a polynucleotide sequence may also be introduced using standard mutagenesis techniques, such as oligonucleotide-directed site-specific mutagenesis. Further, siRNAs may be chemically modified or conjugated with other molecules to improve their stability and/or delivery properties. Included as one aspect of the invention are siRNAs as described herein, wherein one or more ribose sugars has been removed therefrom.

Alternatively, siRNA polynucleotide molecules may be generated by in vitro or in vivo transcription of suitable DNA sequences (e.g., polynucleotide sequences encoding a target polypeptide, or a desired portion thereof), provided that the DNA is incorporated into a vector with a suitable RNA polymerase promoter (such as for example, T7, U6, H1, or SP6 although other promoters may be equally useful). In addition, an siRNA polynucleotide may be administered to a mammal, as may be a DNA sequence (e.g., a recombinant nucleic acid construct as provided herein) that supports transcription (and optionally appropriate processing steps) such that a desired siRNA is generated in vivo.

In one embodiment, an siRNA polynucleotide, wherein the siRNA polynucleotide is capable of interfering with expression of a target polypeptide can be used to generate a silenced cell. Any siRNA polynucleotide that, when contacted with a biological source for a period of time, results in a significant decrease in the expression of the target polypeptide is included in the invention. Preferably the decrease is greater than about 10%, more preferably greater than about 20%, more preferably greater than about 30%, more preferably greater than about 40%, about 50%, about 60%, about 70%, about 75%, about 80%, about 85%, about 90%, about 95% or about 98% relative to the expression level of the target polypeptide detected in the absence of the siRNA. Preferably, the presence of the siRNA polynucleotide in a cell does not result in or cause any undesired toxic effects, for example, apoptosis or death of a cell in which apoptosis is not a desired effect of RNA interference.

In another embodiment, the siRNA polynucleotide that, when contacted with a biological source for a period of time, results in a significant decrease in the expression of the target polypeptide. Preferably the decrease is about 10%-20%, more preferably about 20%-30%, more preferably about 30%-40%, more preferably about 40%-50%, more preferably about 50%-60%, more preferably about 60%-70%, more preferably about 70%-80%, more preferably about 80%-90%, more preferably about 90%-95%, more preferably about 95%-98% relative to the expression level of the target polypeptide detected in the absence of the siRNA. Preferably, the presence of the siRNA polynucleotide in a cell does not result in or cause any undesired toxic effects.

In yet another embodiment, the siRNA polynucleotide that, when contacted with a biological source for a period of time, results in a significant decrease in the expression of the target polypeptide. Preferably the decrease is about 10% or more, more preferably about 20% or more, more preferably about 30% or more, more preferably about 40% or more, more preferably about 50% or more, more preferably about 60% or more, more preferably about 70% or more, more preferably about 80% or more, more preferably about 90% or more, more preferably about 95% or more, more preferably about 98% or more relative to the expression level of the target polypeptide detected in the absence of the siRNA. Preferably, the presence of the siRNA polynucleotide in a cell does not result in or cause any undesired toxic effects.

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

### Genetic Modification

In other related aspects, the invention includes an isolated nucleic acid encoding an inhibitor, wherein the inhibitor preferably an siRNA, inhibits a regulator, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The desired polynucleotide can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means. It is readily understood that the introduction of the expression vector comprising the polynucleotide of the invention yields a silenced cell with respect to a regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

### Modified Cell

In one embodiment, the instant invention provides a cell-based system for expressing an inhibitor that is capable of inhibiting any one or more of ALK, STAT3 or CD274. The invention includes a cell that has been modified to possess a heightened immunogenicity as compared to an otherwise identical cell not modified to have one or more oncogenic protein inhibited. The modified cell is suitable for administration to a mammalian recipient alone or in combination with other therapies.

This invention includes a cell with heighted immunogenicity or otherwise referred to as an antigenic composition. The antigenic composition of the invention is useful as a vaccine. The antigenic composition induces an immune response to the antigen in a cell, tissue or mammal (e.g., a human). In some instances, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

A vaccine of the present invention may vary in its composition of nucleic acid and/or cellular components. In a non-limiting example, a nucleic encoding an antigen might also be formulated with an adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein.

In the context of the present invention, “tumor antigen” or “hyperporoliferative disorder antigen” or “antigen associated with a hyperproliferative disorder” refer to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

In one embodiment, the tumor antigen of the present invention comprises one or more antigenic cancer epitopes immunologically recognized by tumor infiltrating lymphocytes (TIL) derived from a cancer tumor of a mammal.

### Therapeutic Application

The present invention includes an inhibitor of any one or more of ALK, STAT3, CD274, or functional equivalent of any of these proteins. The invention also includes a cell having heighted immunogenicity wherein any one or more of ALK, STAT3 or CD274 in the cells has been inhibited. The immunogenicity of the cell can be measured by monitoring the induction of a cytolytic T-cell response, a helper T-cell response, and/or antibody response to the cell using methods well known in the art.

The present invention includes a method of enhancing the immune response in a mammal comprising the steps of contacting one or more lymphocytes with a cell having heighted immunogenicity, wherein the cell has been modified to have any one or more of ALK, STAT3 or CD274 inhibited in the cell. The cell is a type of vaccine in a mammal. Preferably, the mammal is a human.

Ex vivo procedures are well known in the art and are discussed more fully below. Briefly, cells are isolated from a mammal (preferably a human) and modified to enhance its immunogenicity according to the methods of the invention. For example, the cell is modified to have any one or more of NPM/ALK, STAT3 or CD274 inhibited. The heighted immunogenic cell can be administered to a mammalian recipient to provide a therapeutic benefit. The mammalian recipient may be a human and the cell so modified can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells.

In addition to using a cell-based vaccine in terms of ex vivo immunization, the present invention also provides compositions and methods for in vivo immunization to elicit an immune response directed against an antigen in a patient.

With respect to in vivo immunization, the present invention provides a use of an agent that is capable of inhibiting any one or more of ALK, STAT3 or CD274 as a means to enhance vaccine potency by disabling expression of an immune suppressor in a cell. As such, a vaccine useful for in vivo immunization comprises at least an inhibitor component, wherein the inhibitor component is able to enhance immunogenicity of a cell.

The invention encompasses immunization for cancer and infectious diseases. In one embodiment, the disorder or disease can be treated by in vivo administration of an inhibitor of one or more of ALK, STAT3 or CD274 alone or in combination with an antigen to generate an immune response against the antigen in the patient. Based on the present disclosure, administration of an inhibitor of one or more of ALK, STAT3 or CD274 enhances the potency of an otherwise identical vaccination protocol without the use of an inhibitor of the invention. Without wishing to be bound by any particular theory, it is believed that immune response to the antigen in the patient depends upon (1) the composition administered, (2) the duration, dose and frequency of administration, (3) the general condition of the patient, and if appropriate (4) the antigenic composition administered.

In one embodiment, the mammal has a type of cancer which expresses a tumor-specific antigen. In accordance with the present invention, an antigenic composition can be made which comprises a tumor-specific antigen sequence component. In such cases, the inhibitor of one or more of ALK, STAT3 or CD274 is administered in combination with an immunostimulatory protein to a patient in need thereof, resulting in an improved therapeutic outcome for the patient, evidenced by, e.g., a slowing or diminution of the growth of cancer cells or a solid tumor which expresses the tumor-specific antigen, or a reduction in the total number of cancer cells or total tumor burden.

The disorder or disease can be treated by administration of an inhibitor of one or more of ALK, STAT3 CD274, or their functional equivalents optionally in combination with an antigen (vaccine) to a patient in need thereof. The present invention provides a means to increase immunogenicity of a cell to generate an induced immune response to the tumor-associated antigen in the patient.

In another embodiment, the compounds of the present invention may be used in combination with existing therapeutic agents used to treat cancer. In some instances, the compounds of the invention may be used in combination these therapeutic agents to enhance the antitumor effect of the therapeutic agent.

In order to evaluate potential therapeutic efficacy of the compounds of the invention in combination with the antitumor therapeutics described elsewhere herein, these combinations may be tested for antitumor activity according to methods known in the art.

In one aspect, the present invention contemplates that the inhibitors of the invention may be used in combination with a therapeutic agent such as an anti-tumor agent including but not limited to a chemotherapeutic agent, an anti-cell proliferation agent or any combination thereof.

The invention should not limited to any particular chemotherapeutic agent. Rather, any chemotherapeutic agent can be linked to the antibodies of the invention. For example, any conventional chemotherapeutic agents of the following non-limiting exemplary classes are included in the invention: alkylating agents; nitrosoureas; antimetabolites; antitumor antibiotics; plant alkaloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

There are a variety of antitumor antibiotics that generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

In some embodiments, an effective amount of a compound of the invention and a therapeutic agent is a synergistic amount. As used herein, a “synergistic combination” or a “synergistic amount” of a compound of the invention and a therapeutic agent is a combination or amount that is more effective in the therapeutic or prophylactic treatment of a disease than the incremental improvement in treatment outcome that could be predicted or expected from a merely additive combination of (i) the therapeutic or prophylactic benefit of the compound of the invention when administered at that same dosage as a monotherapy and (ii) the therapeutic or prophylactic benefit of the therapeutic agent when administered at the same dosage as a monotherapy.

### Dosage and Formulation (Pharmaceutical Compositions)

The present invention envisions treating a disease, for example, cancer and the like, in a mammal by the administration of therapeutic agent, e.g. an inhibitor to ALK, STAT3 and/or CD274. In some instances, the therapeutic agent is a cell modified with an inhibitor to ALK, STAT3 and/or CD274 thereby rendering the cell more immunogenic than an otherwise identical cell not modified with the inhibitor.

Administration of the therapeutic agent or modified cell in accordance with the present invention may be continuous or intermittent, depending, for example, upon the recipient's physiological condition, whether the purpose of the administration is therapeutic or prophylactic, and other factors known to skilled practitioners. The administration of the agents or modified cell of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent or modified cell may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

Accordingly, the pharmaceutical composition of the present invention may be delivered via various routes and to various sites in an mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Gene Therapy Administration

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome, aggregated protein or transporter molecule.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

Cells containing the therapeutic agent may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### Screening Agents

The samples used in the detection methods of the present invention include, but are not limited to, cells or tissues, protein, membrane, or nucleic acid extracts of the cells or tissues, and biological fluids such as blood, serum, and plasma. The sample used in the methods of the invention will vary based on the assay format, nature of the detection method, and the tissues, cells or extracts which are used as the sample. Methods for preparing protein extracts, membrane extracts or nucleic acid extracts of cells are well known in the art and can be readily be adapted in order to obtain a sample which is compatible with the method utilized (see, for example, Ausubel et al., Current Protocols in Molecular Biology, Wiley Press, Boston, Mass. (1993)).

One preferred type of sample which can be utilized in the present invention is derived from isolated lymphoma cells. Such cells can be used to prepare a suitable extract or can be used in procedures based on in situ analysis.

Candidate compounds are screened for the ability to inhibit any one or more of ALK, STAT3 or CD274. The determination of the inhibitory function of the candidate agent to any one or more of ALK, STAT3 or CD274 may be done in a number of ways. In any event, the candidate agent should increase the immunogenicity of the cell compare to a cell not contacted with the agent.

The method of identifying an agent capable of inhibiting any one or more of ALK, STAT3 or CD274 includes the initial step of contacting a cell with the agent and determining the activity or level of any one or more of ALK, STAT3 or CD274. A decrease in the activity or level of any one or more of ALK, STAT3 or CD274 indicates that the agent is an inhibitor. Preferably, the agent is also capable of enhancing the immunogenicity of a cell.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The experiments disclosed herein demonstrate that ALK+ T-cell lymphoma (TCL) cells universally express CD274. The CD274 expression is induced by the oncogenic form of ALK tyrosine kinase, chimeric NPM/ALK, through the activation of STAT3, which, in turn, acts as a transcriptional activator of the CD274 gene. These findings identify a novel role of ALK and STAT3 in inducing tumor immune evasion by inducing expression of CD274 and demonstrate for the first time the direct role of an oncogenic protein in controlling the expression of an immunosuppressive cell-surface protein. These observations provide a new rationale to therapeutically target on both functional and expression levels ALK, and STAT3 and their oncogenic functional equivalents to inhibit expression of CD274 and its functional equivalent. Furthermore, they provide strong argument for combining any vaccination-based immunotherapy protocols in cancer with inhibitors targeting an oncogenic protein such as ALK and/or its key signal transmitter(s) such as STAT3.

The materials and methods employed in the experiments disclosed herein are now described.

### ALK+ T-Cell Lymphoma (TCL) and Cutaneous T-Cell Lymphoma (CTCL) Cell Lines and Patients

NPM/ALK-expressing SUDHL-1, JB6, SUPM2, Karpas 299 and L-82 cell lines were derived from ALK+ TCL patients (Zhang et al., 2002, J Immunol 168: 466-474; Marzec et al., 2005, Lab Invest 85: 1544-1554; Marzec et al., 2007, Oncogene 26: 813-821; Kasprzycka et al., 2006, Proc Natl Acad Sci USA 103: 9964-9969; Marzec et al., 2007, Oncogene 26: 5606-5614). IL-2-dependent T-cell line Sez-4 and IL-2 independent MyLa3675 were derived from CTCL patient (Nielsen et al., 1997, Proc Natl Acad Sci USA 94: 6764-6769; Kasprzycka et al., 2008, J Immunol 181: 2506-2512). Jurkat was developed from lymphoblastic T-cell lymphoma. The IL-3-dependent B-cell line BaF3 transfected with an empty vector or vector containing NPM/ALK, either wild type or K210R kinase-deficient mutant (Zhang et al., 2002, J Immunol 168: 466-474; Marzec et al., 2007, Oncogene 26: 5606-5614). The cell lines were cultured at 37° C. and 5% CO2 in the RPMI 1640 medium supplemented with 2 mM L-glutamine, 10% heat-inactivated fetal bovine serum (FBS), 1% penicillin/streptomycin/fungizone mixture, and, where applicable, 200 units of IL-2 (Sez-4) or IL-3 (BaF3).

### Microarray Analysis

The ALK+TCL SUDHL-1 and SUP-M2 cell lines were treated in triplicates with the CEP-14083 ALK inhibitor or the compound's solvent for 4 hour. The isolated RNA was reverse-transcribed, biotin-labeled, and hybridized to the U133 Plus 2.0 array chips (Affymetrix) as described (Marzec et al., 2008, Cancer Res 68: 1083-1091). Microatray data were normalized using MASS algorithm and analyzed using the Partek GS (Partek, St. Paul, Minn.). Differentially expressed genes were identified using ANOVA. Gene list that was estimated to have 5% false discovery rate (FDR=0.05) was used for identification of the NPM/ALK target genes.

### RT-PCR

Total RNA was isolated using RNeasy Mini kit (Qiagen), treated with DNase I (Invitrogen), and reverse-transcribed by using Thermoscript RT-PCR system (Invitrogen) with random hexamers as cDNA synthesis primers. The following primer pairs were used for the cDNA amplification: β-actin, 5′ACCATTGGCAATGAGCGGT′3 (SEQ ID NO: 1) and 5′GTCTTTGCGGATGTCCACGT′3 (SEQ ID NO: 2); CD274, 5′CCTACTGGCATTTGCTGAACGCAT′3 (SEQ ID NO: 3) and 5′ACCATAGCTGATCATGCAGCGGTA′3 (SEQ ID NO: 4). PCR was performed by using Platium TaqDNA polymerase (Invitrogen) for 21 cycles comprised of the denaturation step for 20 seconds at 94° C., annealing for 30 seconds at 58° C. and elongation for 30 seconds at 72° C. The PCR products were visualized by ethium bromide staining in 1.5% agarose gel.

### Immunohistochemical Analysis

Formalin-fixed paraffin-embedded ALK+ TCL tissue specimen slides were heat-treated for antigen retrieval in 10 mM citrate buffer. The sections were blocked with the peroxidase blocking system and incubated at room temperature with rabbit CD274 (B7-H1) antibody (Lifespan Biosciences) at 1:200 dilution for 30 minutes and anti-rabbit-HRP polymer for 30 minutes, washed, exposed to the DABplus chromagen (Dako) for 5 minutes and counterstained with hematoxylin.

### Flow Cytometry

Cells (0.5×106) were washed and stained for 20 minutes with murine antibodies against CD274 (dilution 1:10, clone MIH1, FITC) or CD279 (dilution 1:10, clone MIH4, APC) or FITC- or APC-labeled mouse IgG1 isotype controls. All antibodies were purchased from BD Pharmingen. The stained cells were applied to the flow cytometer (FACSCalibur; Becton Dickinson), and 20,000 events were analyzed. Results of the cell staining are presented as histograms with cell number on the vertical axis and relative fluorescence on the logarithmic horizontal axis.

### Kinase Inhibitors

An ALK potent inhibitor CEP-14083 and its structurally-related, ALK noninhibitory counterpart CEP-11988, both used at the dose of 175 nM, have been described in detail previously (Wan et al., 2006, Blood 107:1617-1623). Inhibitors of: PI3K wortmannin (Calbiochem) used at 20 nM, MEK1/2 U0126 (Promega) used at 15 μM, mTORC1 rapamycin (Cell Signaling Technology) used at 300 nM and Jak3 used at 1 μM as also been described in great detail (Marzec et al., 2005, Lab Invest 85: 1544-1554; Marzec et al., 2007, Oncogene 26: 813-821; Marzec et al., 2007, Oncogene 26: 5606-5614; Marzec et al., 2008, Cancer Res 68: 1083-1091).

siRNA Assay

A mixture of four STAT3 or STAT5b specific siRNA or non-targeting siRNA (all purchased from Dharmacon) was introduced into cells at 100 nM by lipofection with the hew generation Lipofectamine (DMRIE-C; Invitrogen). The procedure was repeated after 24 hours and the cells were cultured for an additional 24 hours. The cells were harvested at one time point 48 hours after first transfection. Extend of the protein knock-down was examined by Flow Cytometry and RT-PCR.

### Electrophoretic Mobility Shift Assay

Nuclear proteins were extracted and incubated with biotin-labeled DNA probes, gel-separated, and transferred to nylon membranes as described (Zhang et al., 2002, J Immunol 168: 466-474; Zhang et al., 2007, Nat Med 13: 1341-1348). Probes used are as follows: 5′-CTTTTTTTATTAATAACA-3′ (SEQ ID NO: 5) and 5′-CGATTTCACCGAAGGTCAG-3′ (SEQ ID NO: 6). These probes correspond to the putative STAT3 binding sites. The blots were developed using the HPR system (Pierce).

### Chromatin Immunoprecipitation Assays

Soluble chromatin-containing lysates obtained from the formaldehyde-fixed and sonicated cells were incubated with STAT3 antibody (Santa Cruz) as described (Kasprzycka et al., 2006, Proc Natl Acad Sci USA 103: 9964-9969; Zhang et al., 2007, Nat Med 13: 1341-1348). Next, the DNA-protein immunocomplexes were precipitated with protein Aagarose beads and the DNA was extracted with phenol/chloroform, precipitated with ethanol and PCR-amplified (Kasprzycka et al., 2006, Proc Natl Acad Sci USA 103: 9964-9969; Zhang et al., 2007, Nat Med 13: 1341-1348) using primers specific for the CD274 gene promoter: 5′-CAAGGTGCGTTCAGATGTTG-3′ (SEQ ID NO: 7) and 5′-GGCGTTGGACTTTCCTGA-3′ (SEQ ID NO: 8).

The results of the experiments presented in this Example are now described.

### Example 1

### ALK+ TCL Cells Express CD274

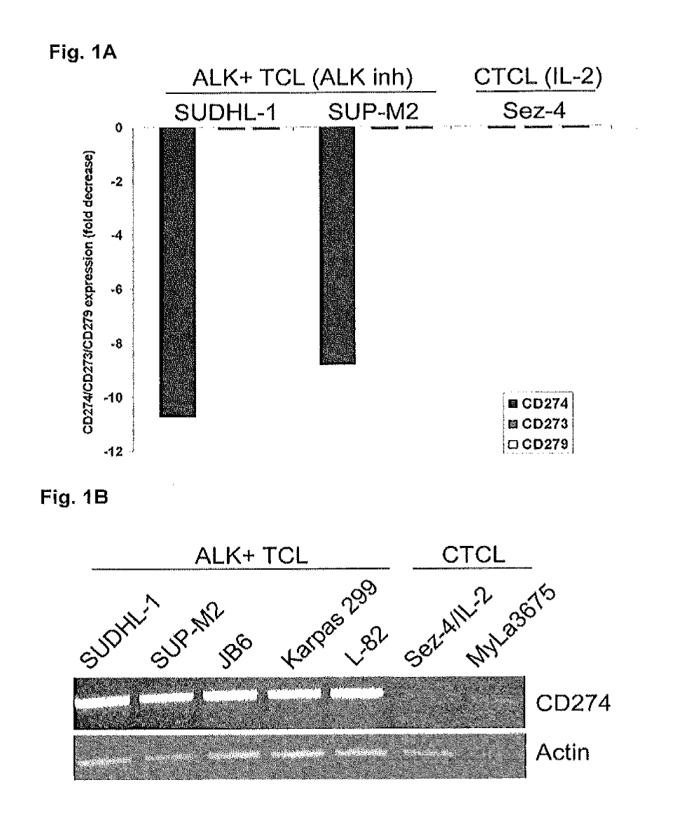

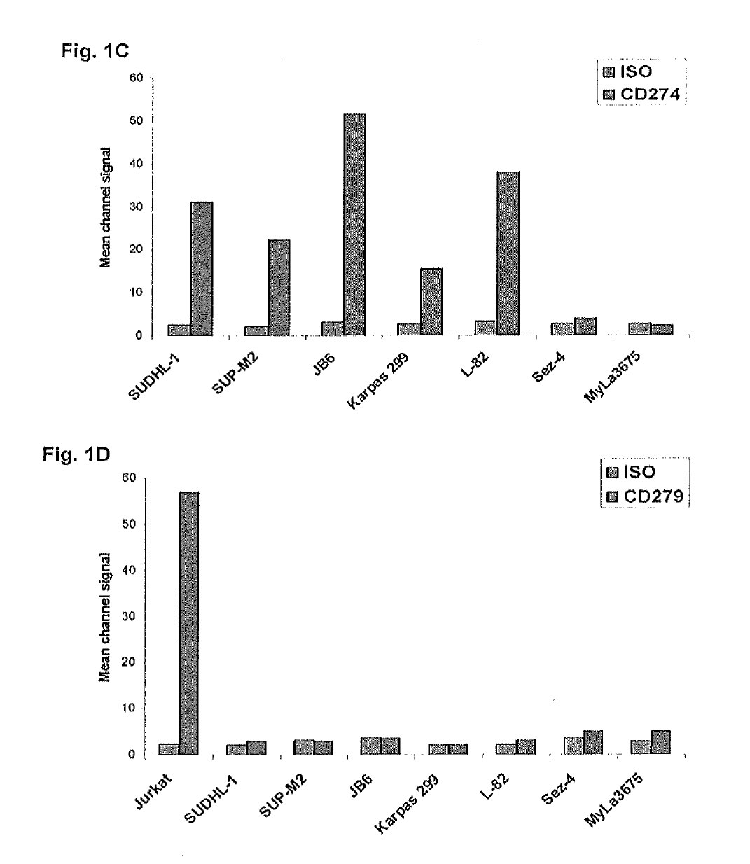

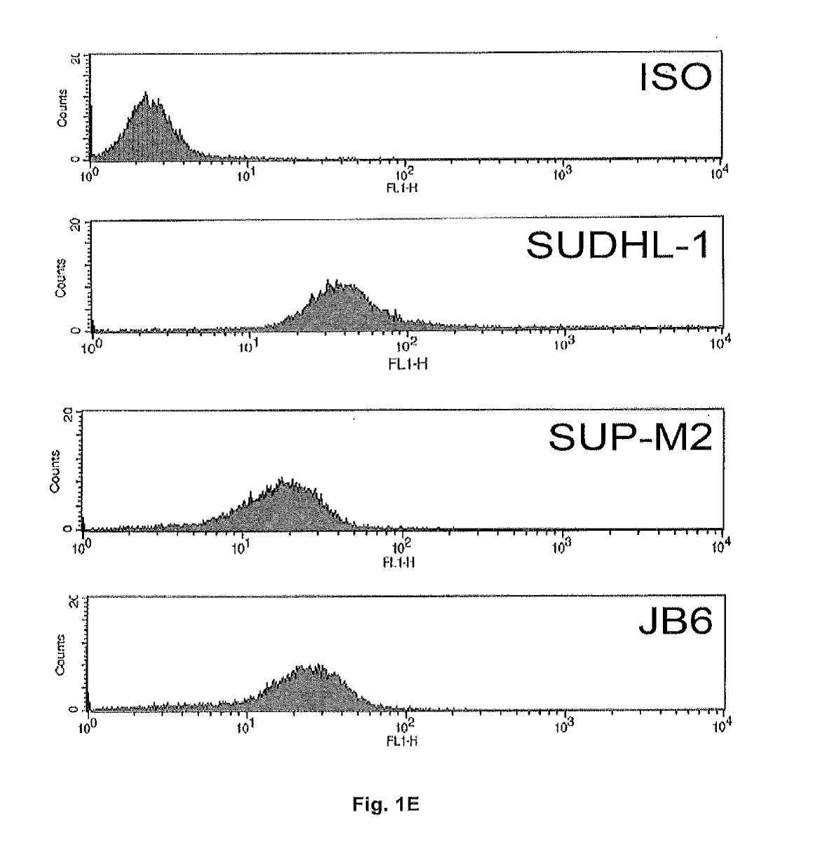

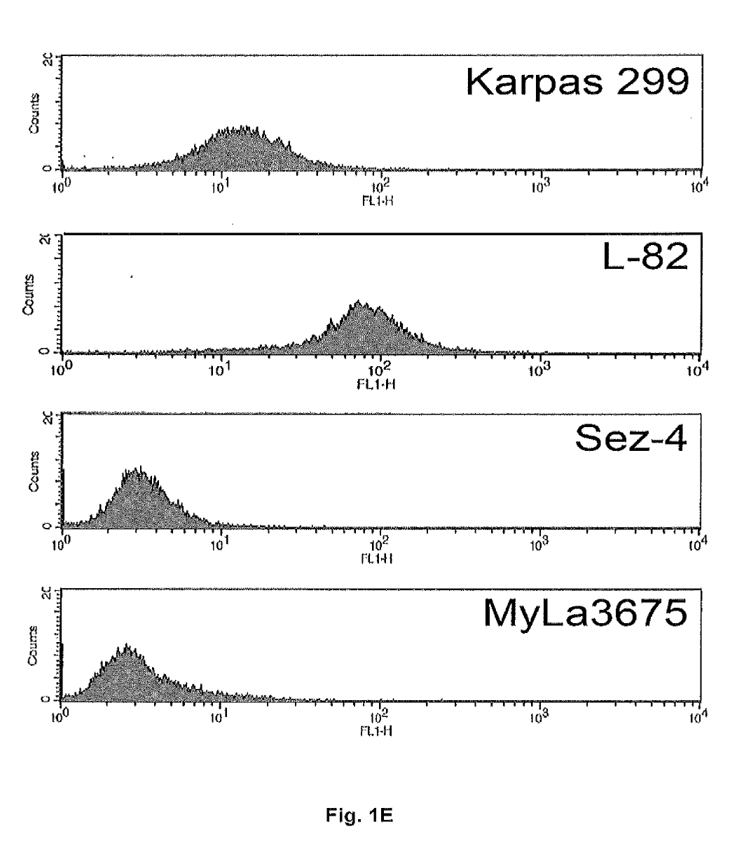

The following experiments were designed to evaluate the mechanisms of NPM/ALK-induced malignant cell transformation. ALK+ TCL cells were screened for changes in gene expression in response to a novel small molecule ALK inhibitor CEP-14083 (Wan et al., 2006, Blood 107:1617-1623) using DNA oligonucleotide array-based genome scale gene expression profiling. When two well-characterized ALK+ TCL-derived cell lines, SUDHL-1 and SUP-M2, (Zhang et al., 2002, J Immunol 168: 466-474; Marzec et al., 2005, Lab Invest 85: 1544-1554; Marzec et al., 2007, Oncogene 26: 813-821; Kasprzycka et al., 2006, Proc Natl Acad Sci USA 103: 9964-9969; Marzec et al., 2007, Oncogene 26: 5606-5614; Zhang et al., 2007, Nat Med 13: 1341-1348) were analyzed, one of the most strongly suppressed genes was the CD274/PD-L1 gene (about 11- and 9-fold decrease in the mRNA expression as compared to the drug vehicle-treated cells; FIG. 1A). No CD274 mRNA expression could be detected in the control, IL-2-dependent and ALK-negative Sez-4 cell line derived from a cutaneous T-cell lymphoma (CTCL) either in the presence or absence of IL-2. In contrast to CD274, no modulation or, for that matter, even expression of the CD274 receptor CD279/PD-1 and the CD274-related ligand CD273/PD-L2 was detected in the ALK+ TCL cell lines SUDHL-1 and SUP-M2 cells which were either treated or untreated with the ALK inhibitor. The CTCL cell line Sez-4 cells also did not express CD279 and CD273, regardless of the IL-2 stimulation status.

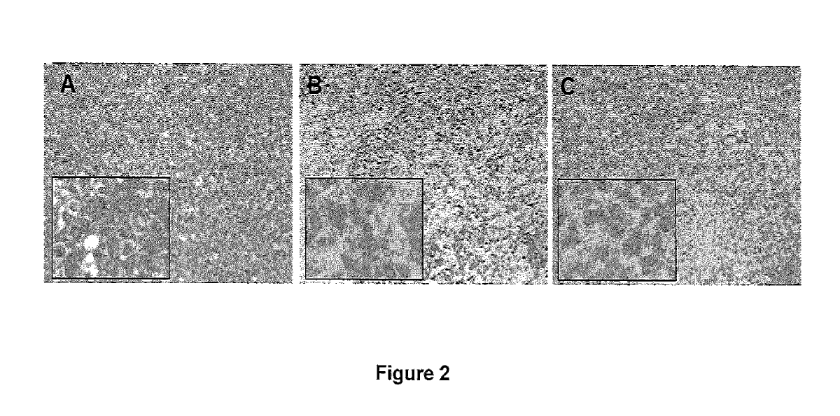

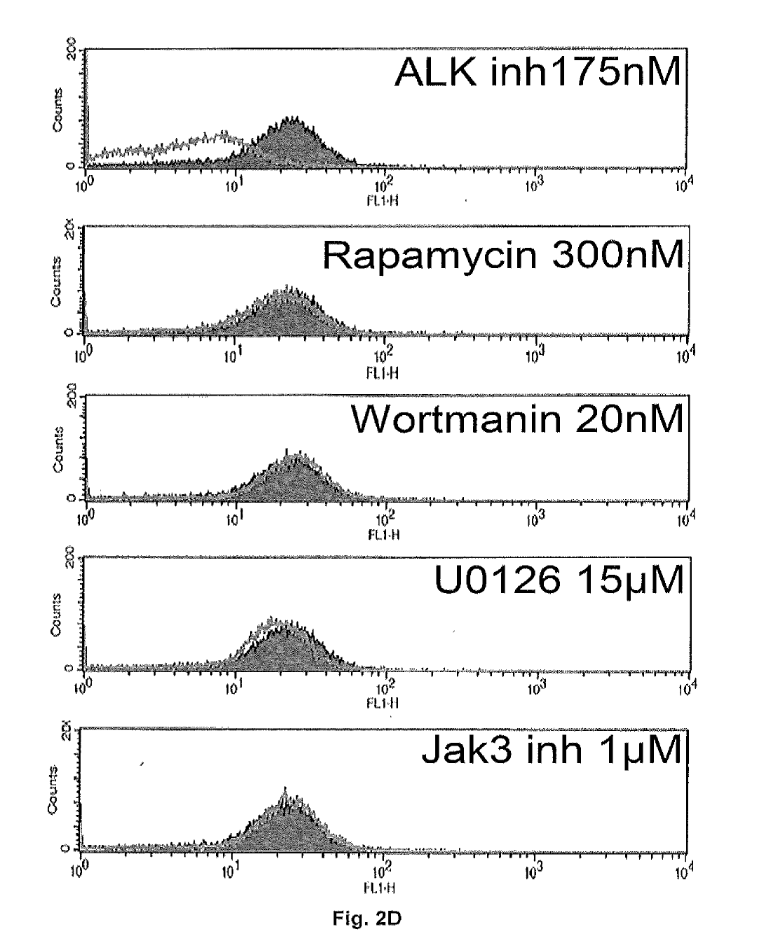

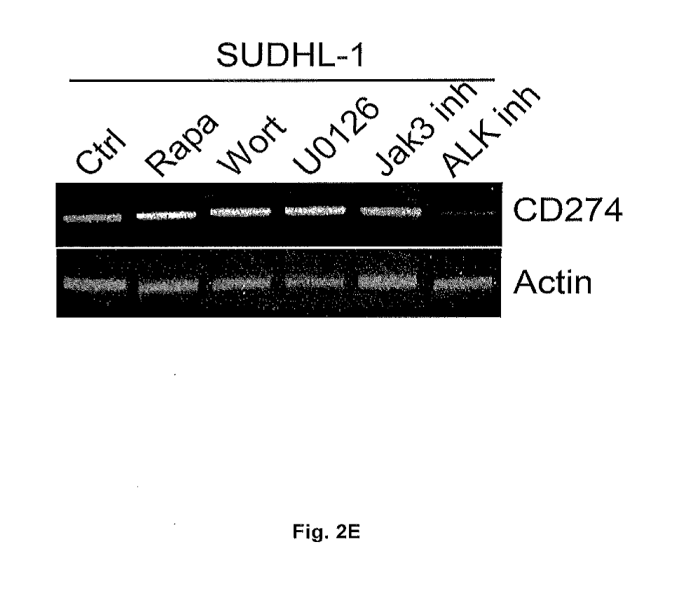

To confirm and expand the finding of CD274 expression by ALK+ TCL cells using a different method, RT-PCR using primers specific for the CD274 cDNA was performed. As shown in FIG. 1B, all five ALK+ TCL cell lines examined strongly expressed CD274 mRNA with only traces of the message seen in the two control CTCL-derived cell lines. CD274 was also strongly expressed by the ALK+ TCL but not CTCL cell lines on the protein level, as demonstrated by flow cytometry analysis (FIG. 1). Of note, none of the cell populations examined expressed CD279 (FIG. 1D). This finding excludes a potential, autologous CD279-CD274 receptor-ligand interaction within the ALK+ TCL cell population. To demonstrate that CD274 expression occurs also in the uncultured, primary ALK+ TCL cells, tissue samples from eighteen cases of ALK+ TCL were examined by immunohistochemistry. Results of this evaluation are shown in FIG. 2 (a representative case). The malignant anaplastic lymphoma cells (FIG. 2A) strongly expressed not only the ALK kinase (FIG. 2B) but also CD274 (FIG. 2C) in all cases examined.

### Example 2

### Expression of CD274 is Induced by NPM/ALK

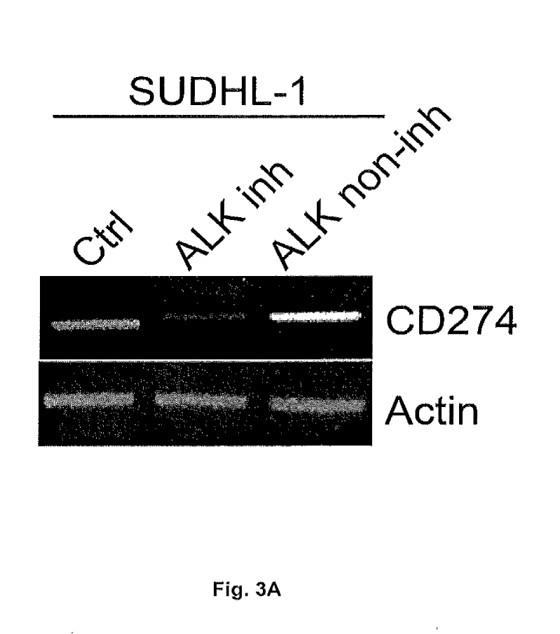

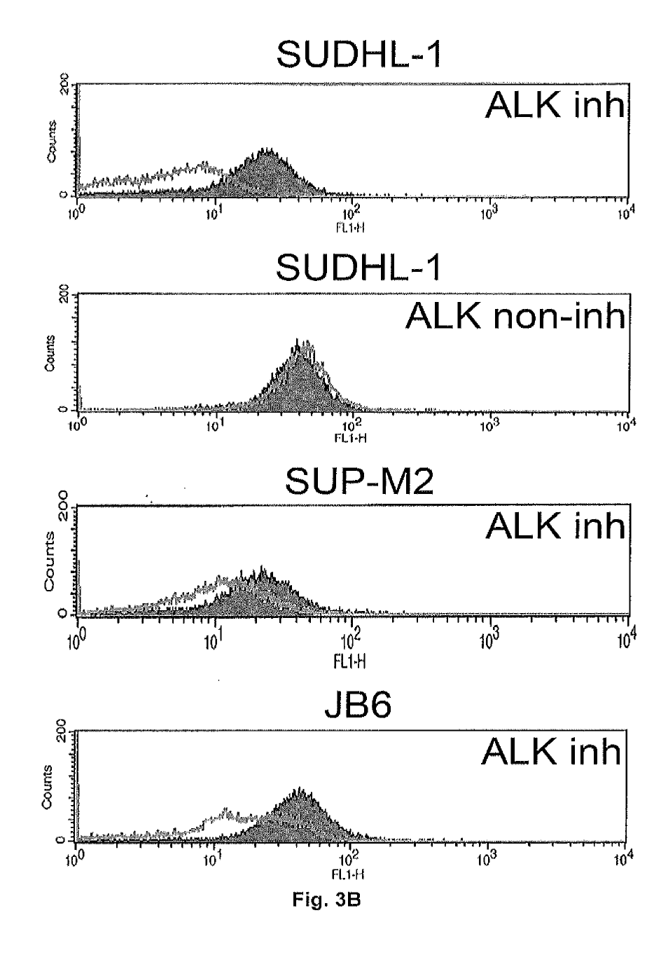

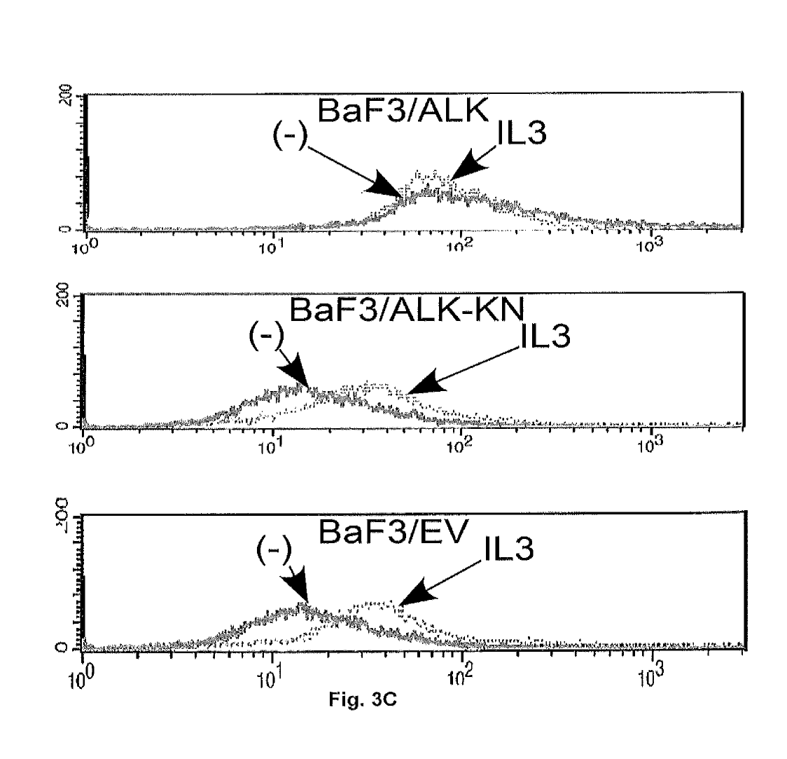

The observation that CEP-14083, the highly potent inhibitor of ALK (Wan et al., 2006, Blood 107:1617-1623), suppressed CD274 mRNA expression in the ALK+ TCL cells as determined by the above-described DNA oligonucleotide array analysis (FIG. 1A) indicated that NPM/ALK induces the expression of CD274. To confirm this finding by a more standard method, RT-PCR was performed using cDNA extracted from cells treated with CEP-14083, or CEP-11988, a structurally closely related analog of CEP-14083 with minimal ALK-inhibitory activity (Wan et al., 2006, Blood 107:1617-1623), or the drug vehicle alone. As shown in FIG. 3A, treatment of the ALK+ TCL SUDHL-1 cell line with CEP-14083 at a pre-selected, rather low, yet strongly ALK inhibitory dose of 175 nM (data not presented), profoundly suppressed the expression of CD274 mRNA. In contrast, treatment with the same dose of CEP-11988 had no effect on CD274 expression. The suppression of CD274 expression in SUDHL-1 cells by CEP-14083 but not CEP-11988 was also detected on the protein level (FIG. 3B). Not surprisingly, CEP-14083 was also effective in inhibiting CD274 protein expression in two other ALK+ TCL cell lines JB6 and SUP-M2 (FIG. 3B).

Because even the most specific kinase domain inhibitors tend to inactivate more than one kinase, the next set of experiments was designed to determined if induced expression of NPM/ALK promotes CD274 expression. To achieve this goal, an IL-3-dependent lymphoid BaF3 cell line after its transfection with a vector containing either the intact NPM/ALK gene, NPM/ALK kinase inactive K210R mutant, or no insert was examined (Zhang et al., 2002, J Immunol 168: 466-474; Kasprzycka et al., 2006, Proc Natl Acad Sci USA 103: 9964-9969; Marzec et al., 2007, Oncogene 26: 5606-5614). Somewhat unexpectedly, BaF3 cells containing all types of constructs expressed CD274 when exposed to IL-3 albeit this expression was consistently higher in the cells containing the intact, wild type ALK (FIG. 3C). More importantly, upon the withdrawal of IL-3 only those BaF3 cells that expressed the intact NPM/ALK were able to completely sustain the CD274 expression, while BaF3 cells expressing NPM/ALK kinase-inactive mutant or no NPM/ALK did not.

### Example 3

### Induction of CD274 Expression by NPM/ALK is Mediated by STAT3

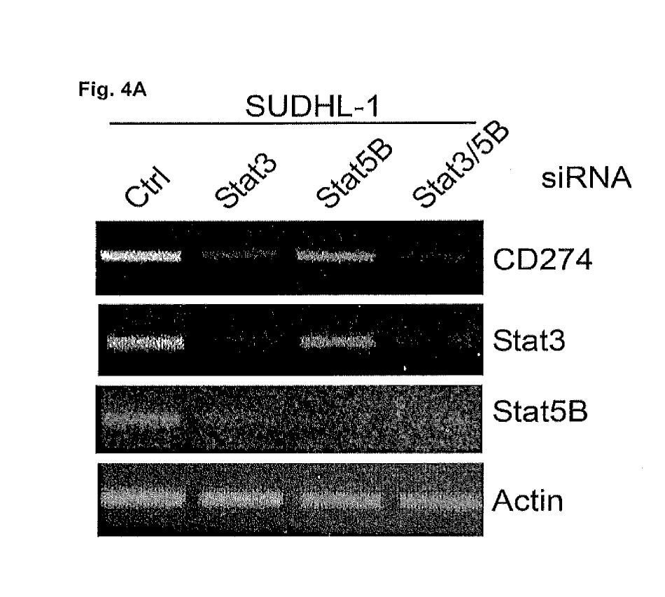

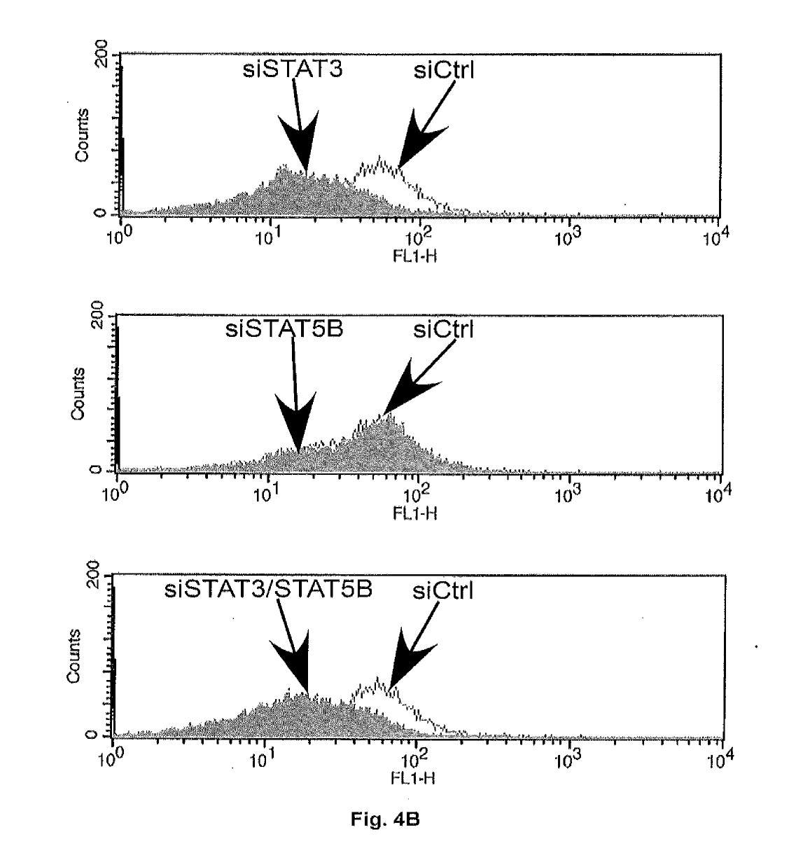

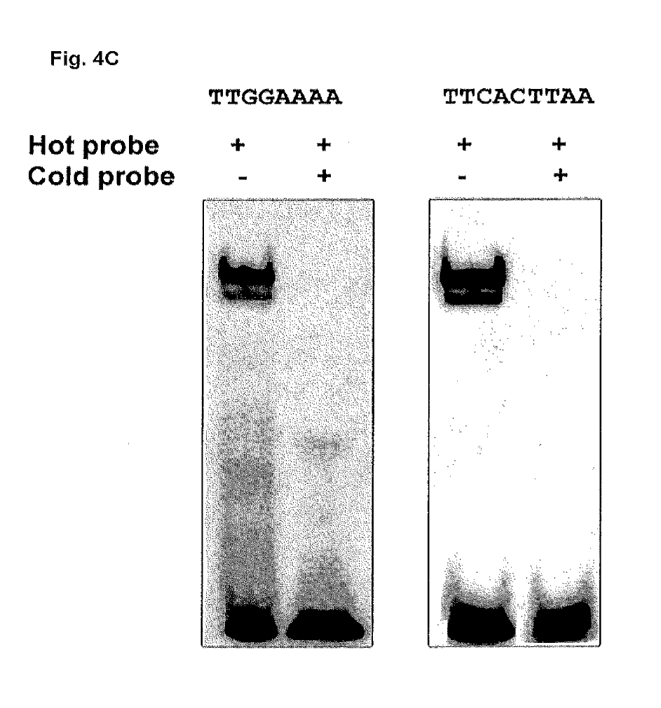

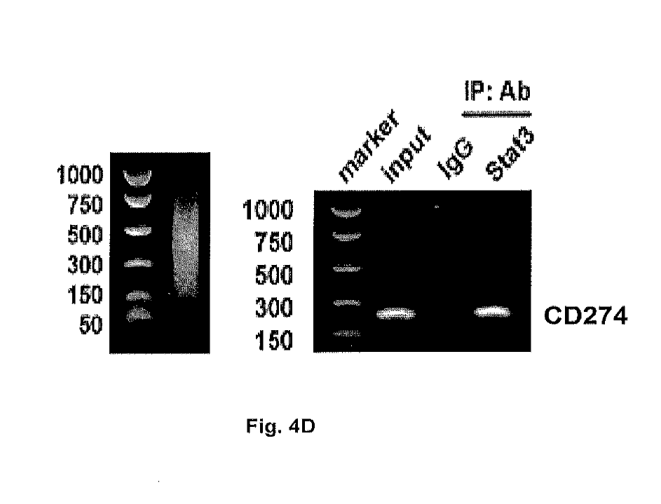

Because the NPM/ALK transforms cells by activating several key signal transducing pathways (Li et al., 2008, Med Res Rev 28: 372-412; Chiarle et al., 2008, Nat Rev Cancer 8: 11-23), the next set of experiments was designed to determine which cell signaling pathways is directly responsible for induction of the CD274 gene transcription. However, treatment of ALK+ TCL SUDHL-1 cells with inhibitors of several kinases known to be down-stream of NPM/ALK: rapamycin (mTORC1 inhibitor), wortmaninn (PI-3K), U0126 (MEK1/2), or Jak3 inhibitor, all used at the pre-selected profoundly inhibitory doses in the ALK+ TCL and CTCL cells (Marzec et al., 2005, Lab Invest 85: 1544-1554; Marzec et al., 2007, Oncogene 26: 813-821; Marzec et al., 2007, Oncogene 26: 5606-5614; Marzec et al., 2008, Cancer Res 68: 1083-1091), had no detectable impact on the CD274 expression either on the protein or mRNA level (FIGS. 2D and 2E, respectively). Faced with this outcome, the next set of experiments was designed to focused on the other potent effectors of the NPM/ALK oncogenicity, STAT3 and STAT5, using siRNA depletion strategy, given the current lack of small molecule inhibitors genuinely selective for STAT3 or STAT5. Depletion in the SUDHL-1 cells of STAT3 but not STAT5, or more specifically STAT5B because SUDHL-1 and other ALK+ TCL cells do not express STAT5a (Zhang et al., 2007, Nat Med 13: 1341-1348), profoundly diminished CD274 expression on both mRNA (FIG. 4a) and protein (FIG. 4b) levels.

The next set of experiments was designed to demonstrate that STAT3 acts as a direct activator of the CD274 gene transcription. First, in silico analysis of the CD274 gene promoter identified four potential STAT3 binding sites was performed. Second, STAT3 binding was evaluated by way of gel electromobility shift assay using two labeled (“hot”) DNA oligonucleotide probes corresponding to the promoter domains containing two of the sites (FIG. 4C). Specificity of binding of both probes was confirmed by its inhibition by the excess of unlabeled, “cold” corresponding probes. Of note, two closely located bands were identified indicating binding of both fast and slow migrating forms of STAT3 (Nielsen et al., 1997, Proc Natl Acad Sci USA 94: 6764-6769). Finally, to demonstrate STAT3 binding to the CD274 gene promoter in vivo, a chromatin immunoprecipitation (ChIP) assay using the PCR primer set capable of amplifying the promoters region containing the STAT3 binding sites was performed. As shown in FIG. 4D, STAT3 did indeed demonstrate strong binding to the CD274 gene promoter.

### Example 4

### Targeting NPM/ALK and STAT3 to Inhibit Suppression of Immune Response Against Malignant Cells

The results presented herein demonstrate that ALK+ TCL cells express a highly immunosuppressive protein, CD274. Further multifaceted analysis revealed that CD274 expression is induced in malignant cells by the chimeric NPM/ALK tyrosine kinase, whose expression resulting from a chromosomal translocation represents the critical oncogenic event in the pathogenesis of ALK+ TCL (Li et al., 2008, Med Res Rev 28: 372-412; Chiarle et al., 2008, Nat Rev Cancer 8: 11-23; Morris et al., 1994, Science 263: 1281-1284; Shiota et al., 1994, Oncogene 9: 1567-1574; Morris et al., 1997, Oncogene 14: 2175-2188; Fujimoto et al., 1996, Proc Natl Acad Sci USA 93: 4181-4186; Bischof et al., 1997, Mol Cell Biol 17: 2312-2325; Kuefer et al., 1997, Blood 90: 2901-2910; Chiarle et al., 2003, Blood 101: 1919-1927).

The results presented herein demonstrate that NPM/ALK induces the CD274 gene activation by activating its key downstream signaling intermediary, the transcription factor STAT3. These findings identify a novel function for NPM/ALK as a promoter of evasion of immune response by inducing CD274 expression and documenting the central role of STAT3 in conferring upon the immunosuppressive phenotype of ALK+ TCL cells. Finally, these observations provide a new rationale to therapeutically target NPM/ALK and STAT3 and provide therapies aimed at boosting immune response against ALK+ TCL cells by inhibiting NPM/ALK or STAT3.

CD274 plays a key role in induction and maintenance of immune tolerance to self-antigens as well as limiting normal immune response against microorganisms to protect the involved tissues from excessive damage incurred during such a response and to prevent its potential autoimmune complications (Dong et al., 2003, J Mol Med 81: 281-287; Okazaki et al., 2007, Int Immunol 19: 813-824). While CD274 has been identified in the whole spectrum of normal hematopoietic and non-hematopoietic cells including macrophages, dendritic cells, activated T and B lymphocytes, endothelial, muscle, and glial cells as well as a large variety of epithelial cells, its expression in such cells is transient and tightly controlled with regard to the exact timing, extent, and specific localization. Several different cytokines produced by immune cells including IFN α, β, and γ, TNFα, IL-2, and IL-17 have been shown to induce or enhance CD274 expression. CD274 is also very commonly expressed by a multitude of malignant cell types of epithelial and hematopoietic cell origin but, in contrast to the normal cells, the expression is persistent. Abundant indirect and less plentiful direct evidence indicates that CD274 plays a key role in induction and maintenance of tolerance towards the malignant cells (Dong et al., 2003, J Mol Med 81: 281-287; Okazaki et al., 2007, Int Immunol 19: 813-824; Keir et al., 2008, Annu Rev Immunol 26:677-704). However, the mechanisms of CD274 induction in such cells remain essentially unknown including lack of any links to genetic changes underlying the very nature of malignant cell transformation.

The results presented herein demonstrate that NPM/ALK oncoprotein induces CD274 expression represents the first example of such a direct link. By its virtue of being constitutively expressed and activated, NPM/ALK secures a persistent, steady supply of the CD274 protein in ALK+TCL cells. In view of NPM/ALK being able to induce expression of IL-10 and TGF-β (Kasprzycka et al., 2006, Proc Natl Acad Sci USA 103: 9964-9969) although not FoxP3 (Kasprzycka et al., 2008, J Immunol 181: 2506-2512), these combined observations indicate that inhibition of immune response against ALK+ TCL is an important component of the NPM/ALK-mediated oncogenicity. Furthermore, NPM/ALK induces expression of these immunosuppressive proteins through its key effector transcription factor STAT3. Given that STAT3 can be activated by a variety of quite diverse tyrosine kinases (Yu et al., 2004, Nat Rev Cancer 4: 97-105), that it is persistently activated in a large spectrum of malignancies, and, that the STAT3 activation plays a key role in oncogenesis (Chan et al., 2004, J Clin Invest 114: 720-728; Ling et al., 2005, Cancer Res 65: 2532-2536; Chiarle et al., 2005, Nat Med 11: 623-629), it is believed that STAT3 is involved in immune evasion of a substantial number of tumors. Of note, STAT3 has also been implicated in down-regulation of immune response in tumors by indirectly inhibiting activation of tumor-infiltrating antigen presenting cells (Wang et al., 2004, Nat Med 10: 48-54) and directly inducting energy in such cells (Cheng et al., 2003, Immunity 19: 425-436).

A few different signaling pathways have been recently implicated in the control of CD274 expression in various types of cells. Accordingly, PI3K/AKT pathway has been found to induce CD274 in the glioma cells by activating mTOR/S6K1 signaling (Parsa et al., 2007, Nat Med 13: 84-88). However, the results presented herein (FIG. S2) as well as those presented in Lee at al. (Lee et al., 2006, FEBS Lett 580: 755-762), who studied lung and hepatocellular carcinoma cell lines, were not able to document the effect of PI3K, mTOR, or MEK/ERK inhibition on the expression of CD274 expression. These findings suggest the existence of alternative signaling pathways, possibly receptor- and tissue-type specific, involved in the control of this important and broadly expressed immunosuppressive protein. The observation that INFα and Toll-like receptor enhance persistent expression of CD274 in malignant plasma cells by acting via MyD88, TRAF6, MAPK signaling pathways (Liu et al., 2007, Blood 110: 296-304) supports this conclusion. Finally, IRS-1 transcription factor has been found to activate the CD274 gene in the lung carcinoma cell line (Lee et al., 2006, FEBS Lett 580: 755-762). Whether IRS-1 and STAT3 act independently or can cooperate in inducing CD274 expression in at least some types of normal and malignant cells remains to be determined.

The results presented herein demonstrate that NPM/ALK induces via STAT3 expression of the CD274 as well as of the immunosuppressive cytokines IL-10 and TGFβ (Kasprzycka et al., 2006, Proc Natl Acad Sci USA 103: 9964-9969). These results provide new rationale for therapeutic inhibition of the kinase or the transcription factor with the former being currently a much more attractive target given the already proven effectiveness of kinase inhibitors in general and the beneficial effects of NPM/ALK inhibition already documented in the preclinical models (Wan et al., 2006, Blood 107:1617-1623; Marzec et al., 2005, Lab Invest 85: 1544-1554; Galkin et al., 2007, Proc Natl Acad Sci USA 104: 270-275). Of note, ALK+TCL patients develop rudimentary humoral (Pulford et al., 2000, Blood 96: 1605-1607) and cellular (Passoni et al., 2002, Blood 99: 2100-2106) immune responses against NPM/ALK but they are per se clearly insufficient to control the tumor growth. In the NPM/ALK-transgene syngeneic mouse transplant model, DNA vaccination with plasmids encoding portions of the cytoplasmic domain of ALK displayed protective effect and significantly enhanced the impact of chemotherapy on survival of the recipient mice (Chiarle et al., 2008, Nat Med 14: 676-680). Therefore, pharmacologically targeting NPM/ALK or STAT3 may drastically increase immunogenicity of the ALK+ TCL cells and, hence, markedly boost the host immune response against the lymphoma cells. Moreover, it may dramatically improve the efficacy of any vaccination protocols targeting ALK or other lymphoma-related antigens. It seems relevant in this context that in the mouse model of renal cell carcinoma, the irradiated cancer-cell vaccine combined with an antibody-induced blockade of CD274 and depletion of regulatory cell-rich CD4+ T cells resulted in complete tumor regression (Webster et al., 2007, J Immunol 179: 2860-2869). This outcome indicates that combination therapy may be required to achieve long-lasting therapeutic effects in human malignancies including ALK+ TCL.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition for enhancing the immunogenicity of a cell, said composition comprising an inhibitor of an oncogenic protein or a down-stream effector protein thereof, wherein said oncogenic protein or down-stream effector protein thereof induces directly or through the effector protein expression of an immunosuppressor.

2. The composition of claim 1, wherein said immunosuppressor is a cell-surface suppressor of immune system response to malignant cells.

3. The composition of claim 1, wherein said oncogenic protein is Anaplastic Lymphoma Kinase (ALK) or an oncogenic functional equivalent thereof capable of inducing expression of an immunosuppressor.

4. The composition of claim 1, wherein said induction of expression of an immunosuppressor is through an ALK down-stream effector, a cell signal transmitter, and the gene trascription activator STAT3 or a functional equivalent thereof.

5. The composition of claim 1, wherein said immunosuppressor is CD274 or a functional equivalent thereof.

6. The composition of claim 1, wherein said inhibitor is selected from the group consisting of a small interfering RNA (siRNA), a microRNA, an antisense nucleic acid, a ribozyme, an expression vector encoding a transdominant negative mutant, an antibody, a peptide and a small molecule.

7. The composition of claim 1, wherein said cell is cancer cell.

8. An isolated cell having inhibited immunogenicity, said cell containing an oncogenic protein or a down-stream effector protein thereof, wherein said oncogenic protein or a down-stream effector protein thereof induces expression of an immunosuppressor.

9. The cell of claim 8, wherein said oncogenic protein is ALK or a functional equivalent thereof.

10. The cell of claim 8, wherein said induction of expression of an immunosuppressor is through STAT3 or a functional equivalent thereof.

11. The cell of claim 8, wherein said immunosuppressor is a cell-surface suppressor of immune system response to malignant cells.

12. The cell of claim 8, wherein said immunosuppressor is CD274 or a functional equivalent thereof.

13. The cell of claim 8, wherein said inhibitor is selected from the group consisting of a small interfering RNA (siRNA), a microRNA, an antisense nucleic acid, a ribozyme, an expression vector encoding a transdominant negative mutant, an antibody, a peptide and a small molecule.

14. The cell of claim 8, wherein said cell is cancer cell.

15. A method of stimulating an immune response in a mammal, said method comprising administering to the mammal an effective amount of a composition comprising an inhibitor of an oncogenic protein or a down-stream effector protein thereof, wherein said oncogenic protein or down-stream effector protein thereof induces directly or through the down-stream effector expression of an immunosuppressor.

16. The method of claim 15, wherein said immunosuppressor is a cell-surface suppressor of immune system response to malignant cells.

17. The method of claim 15, wherein said oncogenic protein is ALK or a functional equivalent thereof.

18. The method of claim 15, wherein said induction of expression of an immunosuppressor is through STAT3 or a functional equivalent thereof.

19. The method of claim 15, wherein said immunosuppressor is CD274 or a functional equivalent thereof.

20. The method of claim 15, wherein said inhibitor is selected from the group consisting of a small interfering RNA (siRNA), a microRNA, an antisense nucleic acid, a ribozyme, an expression vector encoding a transdominant negative mutant, an antibody, a peptide and a small molecule.

21. The method of claim 15, wherein said mammal is suffering from cancer.

22. A method of treating diseases or disorders associated with uncontrolled, abnormal, and/or unwanted cellular activities, the method comprising administering to a mammal in need thereof, a therapeutically effective amount of the compound or the pharmaceutical composition comprising an inhibitor of an oncogenic protein or a down-stream effector thereof, wherein said oncogenic protein or down-stream effector thereof induces directly or through the effector expression of an immunosuppressor.

23. The method of claim 22, wherein said immunosuppressor is a cell-surface suppressor of immune system response to malignant cells.

24. The method of claim 22, wherein said oncogenic protein is ALK or a functional equivalent thereof.

25. The method of claim 22, wherein said induction of expression of an immunosuppressor is through STAT3 or a functional equivalent thereof.

26. The method of claim 22, wherein said immunosuppressor is CD274 or a functional equivalent thereof.

27. The method of claim 22, wherein said inhibitor is selected from the group consisting of a small interfering RNA (siRNA), a microRNA, an antisense nucleic acid, a ribozyme, an expression vector encoding a transdominant negative mutant, an antibody, a peptide and a small molecule.

28. The method of claim 22, wherein said compound or said pharmaceutical composition is administered in combination with a therapeutic agent.

29. The method of claim 28, wherein said therapeutic agent is selected from the group consisting of an anti-tumor agent, a chemotherapeutic agent, an anti-cell proliferation agent, an anti-tumor vaccine and any combination thereof.

30. The method of claim 28, wherein said therapeutic agent is administered simultaneously, prior to, or after administration of said compound.

31. The method of claim 28, wherein said mammal is suffering from cancer.

32. The method of claim 28, wherein said mammal is a human.

33. A method of screening for an inhibitor of an oncogenic protein or a down-stream effector thereof, wherein said oncogenic protein or down-stream effector thereof induces directly or through the down-stream effector expression of an immunosuppressor, said method comprising contacting said inhibitor with a cell and determining the effect of the inhibitor on expression level of CD274 or a functional equivalent thereof.

34. The method of claim 33, further comprising determining the effect of said inhibitor on the cell concentration of CD274 or a functional equivalent thereof.

35. The method of claim 33, further comprising determining the immunogenicity of said cell.

36. A method of diagnosing a disease in a mammal, the method comprising measuring the expression level of CD274 or a functional equivalent thereof from a biological sample derived from said mammal and comparing the expression level of CD274 or a functional equivalent thereof from a biological sample derived from an otherwise identical healthy mammal, wherein an increase in expression level of CD274 is an indication that said mammal has a disease.

37. The method of claim 34, wherein said biological sample is selected from the group consisting of a tumor tissue or a bodily fluid.

38. The method of claim 35, wherein said bodily fluid is peripheral blood or urine.

39. A method of monitoring a response to anti-cancer therapy in a mammal, the method comprising measuring the expression level of CD274 or a functional equivalent thereof from a biological sample derived from said mammal and comparing the expression level of CD274 from a biological sample derived from an otherwise identical healthy mammal, wherein a decrease in expression level of CD274 or a functional equivalent thereof is an indication that said mammal has responded to said therapy.

40. The method of claim 39, wherein said biological sample is selected from the group consisting of a tumor tissue or a bodily fluid.

41. The method of claim 35, wherein said bodily fluid is peripheral blood or urine.


SEQUENCE LISTING (8 sequences)

SEQ ID NO: 1 - DNA, 19 bp, Artificial
  Beta-actin primer
accattggcaatgagcggt

SEQ ID NO: 2 - DNA, 20 bp, Artificial
  Beta-actin primer 2
gtctttgcggatgtccacgt

SEQ ID NO: 3 - DNA, 24 bp, Artificial
  CD274 primer
cctactggcatttgctgaacgcat

SEQ ID NO: 4 - DNA, 24 bp, Artificial
  CD274 primer II
accatagctgatcatgcagcggta

SEQ ID NO: 5 - DNA, 18 bp, Artificial
  probe
ctttttttattaataaca

SEQ ID NO: 6 - DNA, 19 bp, Artificial
  probe II
cgatttcaccgaaggtcag

SEQ ID NO: 7 - DNA, 20 bp, Artificial
  CD274 gene promoter primer
caaggtgcgttcagatgttg

SEQ ID NO: 8 - DNA, 18 bp, Artificial
  CD274 gene promoter primer II
ggcgttggactttcctga